SEQUENCE LISTING (11 sequences)

SEQ ID NO: 1 - DNA, 33 bp, Artificial Sequence
  Description of Artificial Sequence Synthetic oligonucleotide
aaaaactaagaaaaaaaaatggcagtgtcttag

SEQ ID NO: 2 - DNA, 26 bp, Artificial Sequence
  Description of Artificial Sequence Synthetic oligonucleotide
acaaccagctaagacactgccatttt

SEQ ID NO: 3 - DNA, 37 bp, Artificial Sequence
  Description of Artificial Sequence Synthetic oligonucleotide
aaaaataccctttatataaaaataatactgccgggta

SEQ ID NO: 4 - DNA, 27 bp, Artificial Sequence
  Description of Artificial Sequence Synthetic oligonucleotide
tccatcattacccggcagtattatttt

SEQ ID NO: 5 - DNA, 33 bp, Artificial Sequence
  Description of Artificial Sequence Synthetic oligonucleotide
aaaaaacccaaataaaaaataatactgccgggt

SEQ ID NO: 6 - DNA, 23 bp, Artificial Sequence
  Description of Artificial Sequence Synthetic oligonucleotide
tccatcattacccggcagtatta

SEQ ID NO: 7 - DNA, 35 bp, Artificial Sequence
  Description of Artificial Sequence Synthetic oligonucleotide
aaaaactaagaaaaaaaaatggcgcagtgtcttag

SEQ ID NO: 8 - DNA, 26 bp, Artificial Sequence
  Description of Artificial Sequence Synthetic oligonucleotide
acaaccagctaagacactgccatttt

SEQ ID NO: 9 - DNA, 33 bp, Artificial Sequence; identical to SEQ ID NO: 1
  Description of Artificial Sequence Synthetic oligonucleotide

SEQ ID NO: 10 - DNA, 37 bp, Artificial Sequence; identical to SEQ ID NO: 3
  Description of Artificial Sequence Synthetic oligonucleotide

SEQ ID NO: 11 - DNA, 33 bp, Artificial Sequence; identical to SEQ ID NO: 5
  Description of Artificial Sequence Synthetic oligonucleotide

(19) United States
(12) Patent Application Publication
(10) Pub. No.: US 2024/0156993 A1
(43) Pub. Date: May 16, 2024

(54) PLASMONICS SENSING NANOPLATFORMS FOR HUMAN STEM CELL APPLICATIONS AND METHODS THEREOF

(71) Applicants: DUKE UNIVERSITY, Durham, NC (US); THE REGENTS OF THE UNIVERSITY OF CALIFORNIA, Oakland, CA (US)

(72) Inventors: Tuan VO-DINH, Durham, NC (US); Matthias HEBROK, San Francisco, CA (US); Bridget CRAWFORD, Durham, NC (US); Eleonora DE KLERK, San Francisco, CA (US); Hsin-Neng WANG, Durham, NC (US)

(21) Appl. No.: 17/759,729

(22) PCT Filed: Jan. 29, 2021
(86) PCT No.: PCT/US21/15676

§ 371 Date: Jul. 29, 2022

Related U.S. Application Data
(60) Provisional application No. 62/967,143, filed on Jan. 29, 2020.

(51) Int. Cl. A61K 49/00; A61B 5/00; A61K 41/00; C12N 5/071; C12N 15/113; C12N 15/87

(52) U.S. Cl. CPC A61K 49/0097; A61B 5/0071; A61K 41/0047; A61K 49/0054; C12N 5/0676; C12N 15/113; C12N 15/87; A61B 2560/0431; C12N 2310/531; C12N 2320/10; C12N 2506/02

(57) ABSTRACT

A method of monitoring viability of stem cell-derived cells used in stem cell therapy comprises introducing one or more stem cell-derived cells to a cell culture media, introducing one or more nanoprobes to the cell culture media, whereby the one or more stem cell-derived cells are transfected with the one or more nanoprobes, and detecting an optical signal from the one or more nanoprobes after transfection. The method may further comprise introducing the one or more transfected stem cell-derived cells to a subject and detecting the optical signal from the one or more nanoprobes in vivo. The one or more stem cell-derived cells may include a stem cell.

### CROSS REFERENCE TO RELATED APPLICATIONS

This application is a U.S. National Stage Application of International Patent Application No. PCT/US21/15676, filed on Jan. 29, 2021, which claims priority to U.S. Provisional Patent Application No. 62/967,143, filed on Jan. 29, 2020, which are all incorporated by reference herein in their entirety.

### SEQUENCE LISTING

The instant application contains a Sequence Listing which has been submitted electronically in ASCII format and is hereby incorporated by reference in its entirety. Said ASCII copy, created on Nov. 17, 2022, is named 40046_UTIL_SL.txt and is 2,983 bytes in size.

### BACKGROUND

Stem cell technology holds the promise of providing a renewable class of therapeutics for regenerative medicine for a wide variety of diseases. While there have been major advances in the in vitro differentiation of stem cell-derived cells, tissues and organoids, survival of these cells in vivo often poses significant barriers, as the majority of the cells die shortly after transplantation. Currently, the ability to monitor the health state of transplanted cells with non-invasive readouts remains very challenging. To achieve this goal, there is a strong need to develop real-time, practical and efficient, sensing nanoplatforms, systems and methods to monitor the viability, health state and functional capability of transplanted stem cell systems. Furthermore, such a sensing capability will lead ultimately to important advances and improvement of stem cell-based therapy as the sensing nanoplatform can provide a warning signal of cellular non-functionality that will trigger timely remediation procedures and or replacement of the non-functioning stem cells when needed.

### SUMMARY OF THE INVENTION

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

In a first aspect of the invention, an in vivo method of monitoring viability of stem cell-derived cells used in stem cell therapy comprises introducing one or more stem cell-derived cells having one or more nanoprobes to a subject, wherein said one or more nanoprobes are configured to provide health status information for the one or more stem cell-derived cells; and detecting an optical signal from the one or more nanoprobes after introduction of the one or more stem cell-derived cells to the subject. In a feature of this aspect, health status information for the one or more stem cell-derived cells comprises information regarding viability, functioning capability, and/or health state of the one or more stem-cell derived cells.

In a second aspect of the invention, a method of monitoring viability of stem cell-derived cells comprises introducing one or more stem cell-derived cells to a cell culture media, introducing one or more nanoprobes to the cell culture media, whereby the one or more stem cell-derived cells are transfected with the one or more nanoprobes, and detecting an optical signal from the one or more nanoprobes after transfection. In a feature of this aspect, the one or more nanoprobes comprise inverse molecular sentinels (iMS), comprising a plasmonic-active nanoparticle, a stem-loop nucleic acid probe attached at one end to the nanoparticle, the nucleic acid comprising a first sequence (stem-loop probe) and labeled with an optical reporter, and an unlabeled capture placeholder nucleic acid strand comprising a second sequence (placeholder). In another feature of this aspect, transfection comprises electroporating the cell culture media containing the one or more stem cell-derived cells and the one or more nanoprobes; wherein at least a portion of the one or more stem cell-derived cells remain viable after electroporation.

In a third aspect of the invention, a method of transfecting stem cell-derived cells with nanoprobes using electroporation while maintaining a configuration of at least a portion of the nanoprobes comprises providing a cell culture media comprising one or more stem cell-derived cells and one or more nanoprobes having initial configurations, and electroporating the cell culture media containing the one or more stem cell-derived cells and one or more nanoprobes; wherein at least a portion of the one or more nanoprobes maintain their initial configurations after electroporation.

In a fourth aspect of the invention, a method of increasing uptake of nanoprobes into stem cell-derived cells while maintaining viability of the stem cell-derived cells comprises providing a cell culture media comprising one or more stem cell-derived cells and a plurality of nanoprobes, and electroporating the cell culture media containing the one or more stem cell-derived cells and plurality of nanoprobes; whereby an amount of the plurality of nanoprobes transfected into the one or more stem-cell derived cells is greater than an amount that would have transfected into the one or more stem-cell derived cells if transfection consisted of only passive uptake.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments, in which

FIG. 1 is a schematic illustration showing the operating principle of an “inverse Molecular Sentinel” (iMS) detection approach (“Off-to-On” scheme) in accordance with one embodiment of the present disclosure.

FIG. 2 shows the design of iMS nanoprobes for miRNA biomarkers (miR-34a, miR-200-3p, and miR200c-3p) for cell apoptosis in accordance with one embodiment of the present disclosure. Figure discloses SEQ ID NOS 9, 2, 10, 4, 11, and 6, respectively, in order of appearance.

FIG. 3 shows the design of iMS nanoprobes for miRNA biomarkers miR-34a using locked nucleic acids in accordance with one embodiment of the present disclosure. Figure discloses SEQ ID NOS 9 and 8, respectively, in order of appearance.

FIG. 4. is a graph showing the “therapeutic window” in tissue and absorption spectra of biological components.

FIG. 5 are images showing (top) TEM images of nanostars formed under different Ag+ concentrations and (bottom) simulation of the electric field |E| in the vicinity of the nanostars in response to a z-polarized plane wave incident E-field of unit amplitude in accordance with one embodiment of the present disclosure.

FIGS. 6A and 6B are schematic diagrams showing the delivery of transfected stem cell-derived cells in accordance with one embodiment of the present disclosure.

FIGS. 7A and 7B are schematic diagrams showing the monitoring of the health status and functional viability of the transfected stem cell-derived cells using a portable fiberoptics-based Raman diagnostic system (a) in accordance with one embodiment of the present disclosure (b) and a handheld Raman reader.

FIGS. 8A, 8B, and 8C are graphs showing (a) the absorbance spectra of the gold nanostar solutions in citrate in citrate buffer, (b) corresponding finite element method (FEM)-generated absorption spectra, and (c) the scatter plots of polarization-averaged absorption against aspect ratio (AR) tuned by varying branch height while keeping the base width, core, and tip diameters and branch number constant in accordance with one embodiment of the present disclosure.

FIG. 9 is a schematic diagram of gold nanoprobes for intracellular sensing inside a stem cell in accordance with one embodiment of the present disclosure.

FIG. 10 is a graph showing electroporation of INS1 cells with AuNS-PEG in accordance with one embodiment of the present disclosure.

FIGS. 11A and 11B are images showing (A) image of the cells after electroporation; (B) iMS signal remains “OFF” after electroporation in accordance with one embodiment of the present disclosure.

FIGS. 12A and 12B are images showing (A) image of the cells after electroporation; (B) iMS signal remains “ON” after electroporation protocol in accordance with one embodiment of the present disclosure.

FIG. 13 is a graph showing SERS spectrum with iMS “ON” (Upper curve) and iMS probe “OFF” (Lower curve) in accordance with one embodiment of the present disclosure.

FIGS. 14A and 14B are graphs showing (A) SERS spectrum of CEL-miR39 (Cy5) nanoprobes in solution; (B) SERS spectrum of CEL-miR39 (Cy5) in INS1 cells in accordance with one embodiment of the present disclosure.

FIGS. 15A and 15B are graphs showing inductively coupled plasma/mass spectrometry (ICP/MS) quantification of AuNSs in hESCs 48 hours after electroporation (0.15 nM AuNSs, OPTIMEM) in accordance with one embodiment of the present disclosure.

FIGS. 16A and 16B are graphs showing ICP/MS quantification of AuNSs in hESCs right after electroporation (0.15 nM AuNSs, PBS) in accordance with one embodiment of the present disclosure.

FIG. 17 is a series of images showing multiphoton z-stack image of hESCs cells, 24 h after electroporation with 0.15 nM AuNSs in accordance with one embodiment of the present disclosure.

FIG. 18 is a series of flow cytometry images showing hESCs stained with DAPI 30 minutes after electroporation in accordance with one embodiment of the present disclosure.

FIG. 19 is a series of images showing bright field images and fluorescence image of D1, D5 and D20 clusters, respectively (all clusters contain AuNSs) in accordance with one embodiment of the present disclosure.

FIGS. 20A and 20B are graphs showing ICP/MS quantification of AuNSs at different stages of beta cell differentiation in accordance with one embodiment of the present disclosure.

FIGS. 21A and 21B are flow cytometry analyses of D2 spheres electroporated with 0.15-nM AuNSs (A) or 1.5-nM AuNSs in accordance with one embodiment of the present disclosure.

FIGS. 22A and 22B are flow cytometry analyses of D20 spheres electroporated with 0.15-nM AuNSs (A) or 1.5-nM AuNSs (B), in accordance with one embodiment of the present disclosure.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations when interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder, or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e., living organism, such as a patient). In some embodiments, the subject comprises a human who is undergoing a procedure with the compositions, systems, and methods described herein.

As used herein, the term “transfection” refers to the process of delivering, passively or actively, nucleic acids, small proteins, and other particles (e.g., nanoprobes as described herein) into eukaryotic cells. Delivery can be achieved by using any number of known means, including but not limited to, passive uptake, chemical transfection reagents, electroporation (gene electrotransfer), viral transduction, and the like.

As used herein, the term “administering” or “administration” refers to the location and/or route and/or path by which a nanoprobe, a transfected stem cell-derived cell, and/or the like according to the present disclosure is introduced to a subject. Suitable forms of administration include, but are not limited to, intradermal injection, subcutaneous injection, intramuscular injection, intravenous injection, intraperitoneal injection, intracavitational injection (e.g., injection into a pre-existing physiologic or pathologic body cavity), oral, anal, inhalational, nasal spray, and dermal patch. One skilled in the relevant art can easily select the route most likely to be a therapeutically effective modality for a particular agent. In some embodiments, the route of administration comprises subcutaneous injection.

Unless otherwise defined, all technical terms used herein have the same meaning as commonly understood by one of ordinary skill in the art to which this disclosure belongs.

The present disclosure describes various compositions, systems, methods, and instrumentation using the concept of in vivo plasmonics sensing (IPS) nanoplatforms for stem cell applications. The sensing principle is based on enhancement mechanisms of the electromagnetic field effect. The disclosure presents specific novel and unique concepts of enabling IPS probe technologies for use with stem cell technologies for in situ and in vivo sensing (e.g., implanted sensors), regenerative medicine (e.g., stem cells), and other biomedical applications. Novel aspects include novel nanobiosensors, the combination of the IPS nanoplatform with stem cells, the combination of the IPS plus a stem cell sensing system, portable fiberoptics-based readout systems (e.g., at point of care), and the combination of the IPS plus a stem cell sensing system and handheld readout system (e.g., at home or at the point of need).

### 1. Compositions

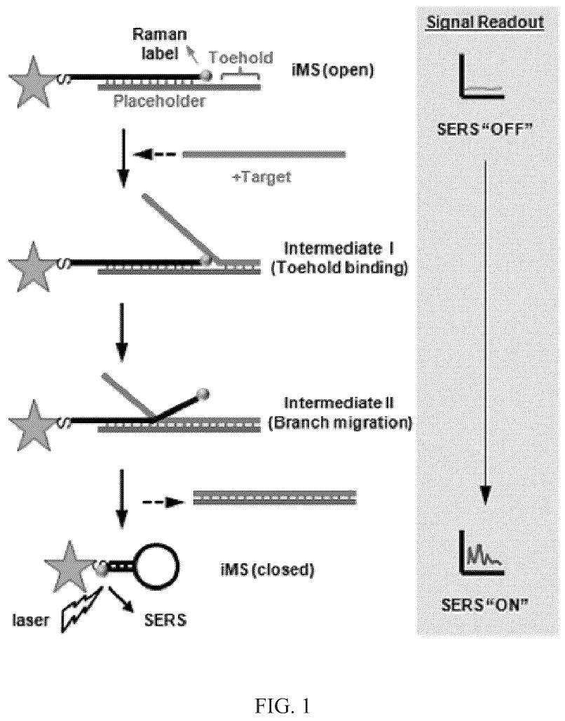

The in vivo plasmonics sensing probe disclosed herein uses a nucleic acid detection scheme that can be referred to as an “Inverse Molecular Sentinel”, or iMS (see, e.g., U.S. patent application Ser. No. 14/861,353, which is incorporated by reference herein in its entirety). An example approach is schematically illustrated in FIG. 1, which shows a schematic design of iMS using a gold nanostars platform.

Generally, and as shown in FIG. 1, an iMS nanoprobe according to the present disclosure comprises (1) at least one plasmonic-active nanoparticle; (2) a stem-loop nucleic acid probe attached at one end to the nanoparticle and labeled with a Raman-active reporter; and (3) an unlabeled capture placeholder nucleic acid strand complementary to target sequence.

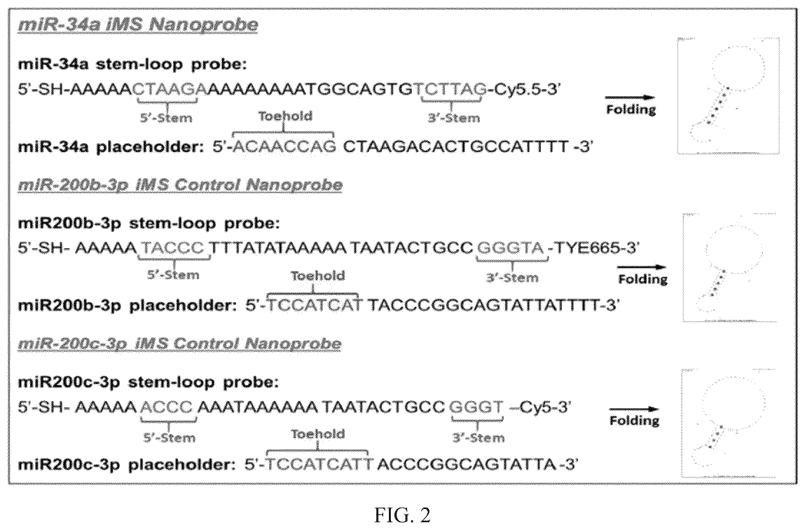

Referring again to FIG. 1, in such embodiments, the “stem-loop” probe of the iMS, having a Raman label at one end of the stem, is immobilized onto a metallic nanoparticle or nanostar via a metal-thiol bond. A partially complementary DNA probe, serving as a “placeholder” strand bound to the iMS nanoprobe, keeps the Raman dye away from the nanostar surface. In the absence of the target, the probe is “open” with very low SERS signal (i.e., the ‘Off’ state) because the plasmon field enhancement decreases significantly with increasing distance from the metallic surface. Upon exposure to the target sequences, the placeholder capture strand leaves the “open” stem-loop probe based on placeholder base-pairing with the complementary target strand following a non-enzymatic strand-displacement process. The target first binds to the toehold region (intermediate I) and begins displacing the nucleic acid probe from the placeholder via branch migration (intermediate II), and finally releases the placeholder from the nanoparticle system. This allows the stem-loop to “close” and moves the Raman label onto the plasmonics-active metal surface. This yields a strong SERS signal, and is thus denoted ‘On’ status. The iMS probe can be designed to detect a variety of targets (DNA, mRNA, RNA, miRNA). A few example miRNA designs are provided below in FIG. 2.

Referring to FIG. 2, in one embodiment, the first sequence (stem-loop probe) comprises the nucleotide sequence AAAAACTAAGAAAAAAAAATGGCAGTGTCTTAG (miR-34a stem loop probe; SEQ ID NO: 1) and the second sequence (placeholder) comprises the nucleotide sequence ACAACCAGCTAAGACACTGCCATTTT (miR-34a placeholder; SEQ ID NO: 2) for miR-34a miRNA sensing.

In another embodiment, the first sequence (stem-loop probe) comprises the nucleotide sequence AAAAATACCCTTTATATAAAAATAATACTGCCGGGTA (miR-200b-3p stem-loop probe; SEQ ID NO: 3) and the second sequence (placeholder) comprises the nucleotide sequence TCCATCATTACCCGGCAGTATTATTTT (miR200b-3p placeholder; SEQ ID NO: 4) for miR200b-3p miRNA sensing.

In another embodiment, the first sequence (stem-loop probe) comprises the nucleotide sequence AAAAAACCCAAATAAAAAATAATACTGCCGGGT (miR200c-3b stem-loop probe; SEQ ID NO: 5) and the second sequence (placeholder) comprises the nucleotide sequence TCCATCATTACCCGGCAGTATTA (miR200c-3p; SEQ ID NO: 6) for miR200c-3p miRNA sensing.

In such embodiments, iMS generates a Raman signal in the presence of the target, based on a non-enzymatic strand-displacement process followed by a conformational change of the hairpin oligonucleotide probe. Upon exposure to the target, the target first binds to an overhang region of the probe-placeholder duplex, then begins displacing the stem-loop probe from the placeholder through a branch migration process, finally releasing the placeholder from the nanobiosensor. The stem-loop structure then closes, moving the Raman label onto the plasmonics-active gold nanoparticle surface, producing a strong SERS signal. As different labels can be bound to the stem-loop probe, multiple targets can be detected simultaneously, maintaining specificity and sensibility.

Notably, and within the scope of another embodiment, the nanobiosensors can have a dual role, serving as target miRNA trap. Once the placeholder traps a target miRNA, the miRNA is no longer functional, and its effects on gene expression are blocked.

In another embodiment, the nanoprobes are further modified to achieve sensitive detection, i.e., low limit of optical detection, or LOD, that produce low background in the absence of targets. In an example embodiment, the physical length of the Raman-labeled stem-loop probe is designed to be around 10 nm for a low background SERS signal in its OFF state. This distance is designed to keep the Raman label away from the gold nanostars (i.e., to have weak or no “lightning rod” effect). Because of the short sequence lengths of miRNAs make it difficult to design stem-loop probes with the desired length, a spacer sequence of around 10 nucleotides is added between the loop and the 5′-end stem. This spacer is used to increase the physical length of the probe to keep the Raman label at an appropriate distance away from the nanostar surface while hybridizing to a placeholder strand. Importantly, this “internal” spacer does not affect the generation of a strong SERS signal when the probe is in a closed stem-loop configuration.

In another embodiment, a short poly(T) tail (4 thymine bases) is added to the 3′-end of the placeholder strand (i.e., hybridizing to a portion of the internal spacer in the probe) to minimize the background signal without affecting the sensor functionality. The addition of the poly(T) tail reduces the strong SERS background signal by enhancing the hybridization efficiency between the placeholder and stem-loop probe.

In another aspect, the present disclosure provides for an intracellular iMS nanoprobe comprising, consisting of, or consisting essentially of locked nucleic acids and nucleases-resistant modifications that can increase duplex stability and prevent degradation, respectively. LNA is a conformationally restricted nucleic acid analogue, in which the ribose ring is locked into a rigid C3′-endo (or Northern-type) conformation by a simple 2′-O, 4′-C methylene bridge. LNA has many attractive properties, such as high binding affinity, excellent base mismatch discrimination capability, and likely decreased susceptibility to nuclease digestion. Duplexes involving LNA (hybridized to either DNA or RNA) display a large increase in melting temperatures ranging from +3.0° C. to +9.6° C. per LNA modification, compared to corresponding unmodified reference duplexes. Furthermore, LNA oligonucleotides can be synthesized using conventional phosphoramidite chemistry, allowing automated synthesis of both fully modified LNA and chimeric oligonucleotides such as DNA/LNA and LNA/RNA. Other advantages of LNA include its close structural resemblance to native nucleic acids, which leads to particularly good solubility in physiological conditions and easy handling. In addition, owing to its charged phosphate backbone, LNA is nontoxic. All these properties are highly advantageous for a molecular tool for diagnostic applications.

When oligonucleotides are used in cell culture or in vivo experiments—such as in antisense and RNAi applications, and in ribozyme technology, degradation by nucleases is a concern. When used for intracellular analyses, nucleic acid probes tend to generate dramatic false-positive signals due to nuclease degradation, protein binding and thermodynamic fluctuations. Oligonucleotide stability is typically crucial to these types of studies, yet unmodified DNA and RNA oligonucleotides are quickly digested in vitro and in vivo by endogenous nucleases. Multiple endo- and exonucleases exist in vivo. For example, it has been reported that unmodified phosphodiester oligonucleotides may possess a half-life as short as 15-20 min in living cells. In serum, the bulk of biologically significant nucleolytic activity occurs as 3′ exonuclease activity, while within the cell, nucleolytic activity is affected by both 5′ and 3′ exonucleases. To limit nuclease sensitivity, different modifications are substituted.

First, the phosphorothioate (PS) bond substitutes a sulfur atom for a non-bridging oxygen in the phosphate backbone of an oligonucleotide. Approximately 50% of the time (due to the two resulting stereoisomers that can form), PS modification renders the inter-nucleotide linkage more resistant to nuclease degradation. Therefore, in accordance with one embodiment of the present disclosure, at least 3 PS bonds at the 5′ and 3′ oligonucleotide ends are included to inhibit exonuclease degradation.

A naturally occurring post-transcriptional modification of RNA 2′OMe is found in tRNA and other small RNAs. Oligonucleotides can be directly synthesized to contain 2′OMe. This modification prevents attack by single-stranded endonucleases, but not exonuclease digestion. Therefore, it is important to end block these oligos as well. DNA oligonucleotides that include this modification are typically 5- to 10-fold less susceptible to DNases than unmodified DNA. The 2′OMe modification increases stability and binding affinity to target transcripts.

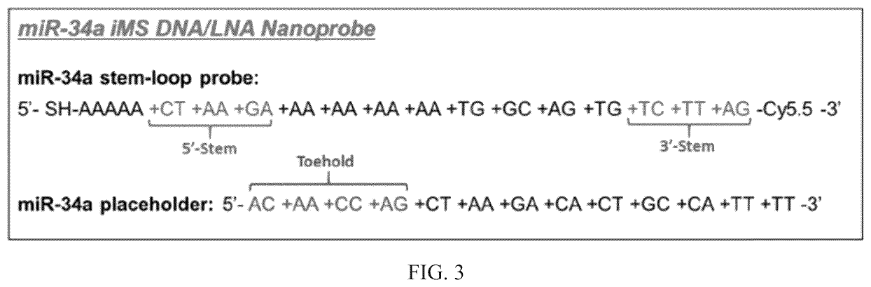

In one embodiment, the present disclosure provides an miR-34a iMS DNA/LNA nanoprobe as shown in FIG. 3 (with LNA indicated by “+”).

This iMS allows for a longer lifetime of sensors within cells. It has been shown that sequences with DNA/LNA alternating bases or all LNA bases were able to resist nonspecific protein binding and impair DNase I digestion. Additionally, a sequence consisting of a DNA stretch less than three bases between LNA bases was able to block RNase H function. Referring again to FIG. 3, a six base-pair stem and alternating DNA/LNA bases is useful for intracellular applications as it ensures reasonable hybridization rates, reduces protein binding, and resists nuclease degradation for both target and probes.

Various plasmonic-active nanoparticles can be used with the iMS nanoprobes as provided herein to yield intense SERS signal of the label at different plasmon resonance wavelengths. Accordingly, in some embodiments the nanoparticles may include, but are not limited to, silver nanospheres, gold nanospheres, nanoshells, nanostars, and the like.

Moreover, various optical reporters can be used with the iMS nanoprobes as provided herein. In some embodiments, optical reporter is selected from the group consisting of: Raman dye, 3,3′-Diethylthiadicarbocyanine iodide (DTDC), 3,3′-diethylthiatric arbocyanine iodide (DTTC), 1,1′,3,3,3′,3′-Hexamethylindotricarbocyanine iodide (HITC), CY3 dye, CY3.5 dye, CY5 dye, CY5.5 dye, CY7 dye, CY7.5 dye, a positively-charged hydrophobic near infrared (NIR) dye, IR-780, IR-792, IR-797, IR-813, methylene blue hydrate (MB), 4-mercaptobenzoic acid (4-MBA), 5,5′-dithiobis-2-nitrobenzoic acid (DTNB), 4-aminothiophenol (4ATP), fluorescein, fluorescein isothiocyanate (FITC), thionine dyes, rhodamine-based dye, crystal violet, a fluorescence label, or absorbance label.

In practice, the thiolated SERS reporter-strand has a Raman dye at one end as the reporter, and a thiol group at the other end for attaching to the nanoparticle. The reporter strand has four segments: stem-L, stem-R, spacer, and placeholder. The stem-L and stem-R segments allows the stem-loop structure to form after the placeholder-strand binds to the target molecule and leaves the nanoconstruct. The spacer segment is designed to provide sufficient distance (e.g., over 10 nm) between the Raman dye and nanoparticle surface to reduce background SERS signal when the probe is open. The placeholder segment (e.g., 8-15 nucleotides) binds to the placeholder-strand to prevent the formation of the stem-loop structure. The placeholder-strand has two segments: placeholder-C and targeting region. The placeholder-C segment is complementary to the placeholder segment of the reporter-strand and to the target sequences. The targeting region (20-30 nucleotides) is complementary to the target sequences.

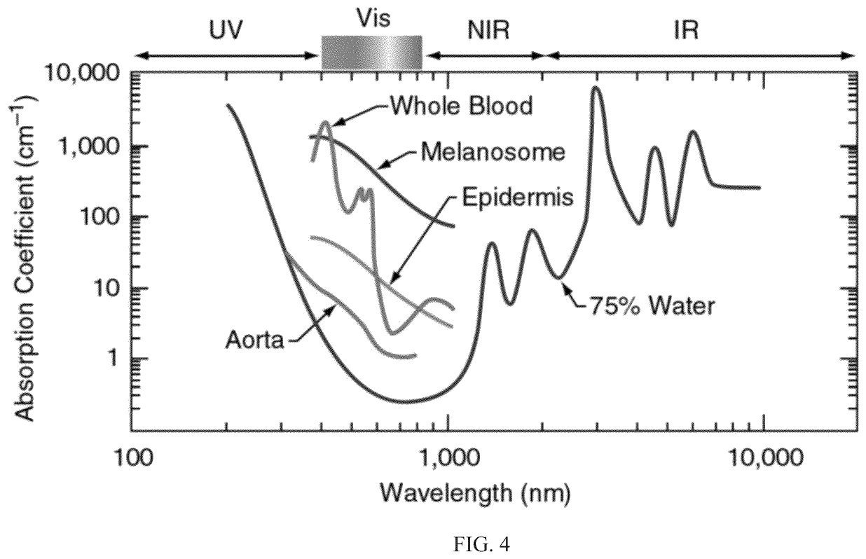

Light having wavelengths within the so-called “therapeutic window” can be used to sense and monitor the viability, health state, and functional capability of transplanted stem cells. The ability of light to penetrate tissues depends on absorption. Within the therapeutic window (or “diagnostic window”), most tissues are sufficiently weak absorbers to permit significant penetration of light. This window extends from approximately 600 nm to 1300 nm, from the orange/red region of the visible spectrum into the near-infrared (NIR). At the short-wavelength end, the window is bound by the absorption of hemoglobin, in both its oxygenated and deoxygenated forms. The absorption of oxygenated hemoglobin increases approximately two orders of magnitude as the wavelength shortens in the region around 600 nm. At shorter wavelengths many more absorbing biomolecules become important, including DNA and the amino acids tryptophan and tyrosine. At the infrared (IR) end of the window, penetration is limited by the absorption properties of water. Within the therapeutic window, scattering is dominant over absorption, and so the propagating light becomes diffuse, although not necessarily entering the diffusion limit. FIG. 4 shows a diagram of the optical window of tissue or therapeutic window in tissue.

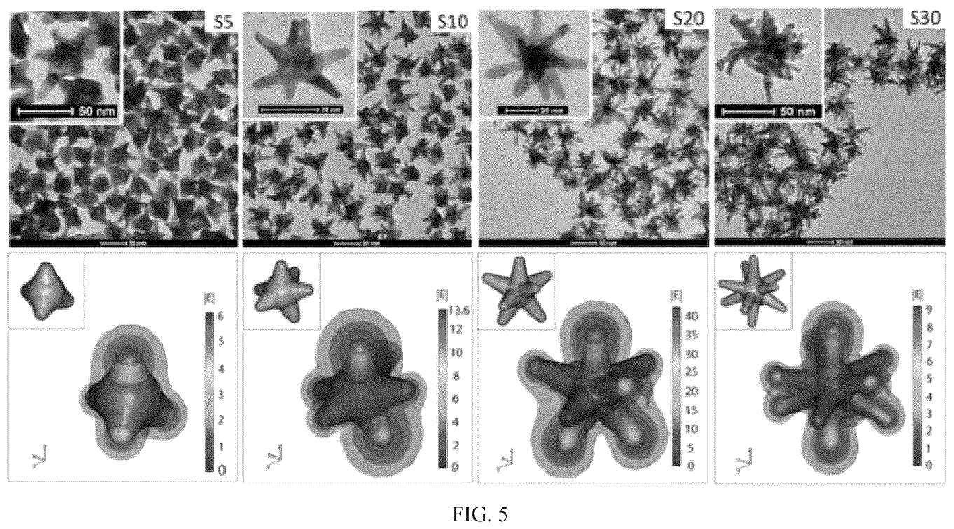

In some embodiments, the plasmonic-active nanoparticle comprises a gold nanostar. Gold nanostars (GNS) are of particular interest as they offer a wide range of optical tunability by making subtle changes to their geometry. The multiple sharp branches on GNS create a “lightning rod” effect that enhances local electromagnetic (EM) field dramatically. This unique tip-enhanced plasmonic property can be tuned in the near infrared (NIR) diagnostic therapeutics optical window, where photons travel further in tissue. In some embodiments plasmon tunability is achieved by adjusting the Ag+ concentration. Specifically, higher concentrations of Ag+ progressively red-shift the plasmon band by forming longer, sharper, and more numerous branches. This is illustrated, for example, in FIG. 5, Top. Nanostar S5 consists of a few protrusions, while S30 comprises multiple long, sharp branches that appear to branch even further. Simulation of |E| in the vicinity of the nanostars in response to a z-polarized plane wave incident E-field of unit amplitude, propagating in the y-direction, and with a wavelength of 800 nm, shows the plasmonic enhancement at the tips of the nanosat, i.e., “lightning rod” effect (see, e.g., FIG. 5, Bottom).

In FIG. 5, the top images are TEM images of nanostars formed under different Ag+ concentrations (S5: 5 μM, S10: 10 μM, S20: 20 μM, S30: 30 μM). The bottom images Simulation of |E| in the vicinity of the nanostars in response to a z-polarized plane wave incident E-field of unit amplitude, propagating in the y-direction, and with a wavelength of 800 nm. The insets depict the 3-D geometry of the stars. The diagrams are not to scale.

In embodiments, GNSs can be synthesized in a controlled fashion and used for deep tissue NIR excitation and absorption in the diagnostic window for stem cell applications. The nanostars' plasmon peaks are tunable, for example, from 600 nm to 1000 nm.

Plasmonic gold nanostars (AuNS) can be synthesized for in vivo use. AuNS of varying sizes can be synthesized by known methods, which includes adding AgNO₃ (along with other components) during synthesis. The synthesis is rapid, reproducible and does not require a polymer as surfactant. Nanostar sizes can be tuned based on concentrations of components used during synthesis. Silver ions play a role in controlling the formation of star geometry. Without Ag+, the resulting particles are polydisperse in both size and shape. Addition of a small amount of Ag+ can aid is synthesizing monodisperse star-shaped particles. Nanoparticle diameters can be within a range of approximately 50-70 nm. Without being bound by theory, it is believed that Ag+ do not form Ag branches but rather assist the anisotropic growth of Au branches on certain crystallographic facets on multi-twinned citrate seeds.

### 2. Applications for Stem Cell Monitoring and Therapy

Stem cells are defined as cells that have the ability to divide for indefinite periods and have the potential to develop into many different specialized cell types that are then used to build tissues and organs. When a stem cell divides into two daughter cells, each daughter cell has the potential to remain a stem cell, meaning an unspecialized cell able to self-renew, or to become any of the specialized cells that constitute the human body, such as neurons, muscle cells, blood cells, endocrine cells and so on.

Stem cells have a crucial role both during the development of the embryo and in adult tissues. Following fertilization, mammalian embryos undergo a series of cell divisions to form an aggregate of cells called morula. Further cell divisions convert the morula into a blastocyst, a cyst-like structure with an inner cavity. The cells localized in this inner cavity, also known as cells of the inner cell mass (ICM), give rise to the entire organism by differentiating into all types of cells that form all tissues and organs. Isolation of ICM cells and culture outside the body resulted in the establishment of human embryonic stem cells (hESCs).

Some stem cells remain undifferentiated until adult age. Adult stem cells have been identified in many organs and tissues, including, but not restricted to, bone marrow, mesenchyme, blood, skeletal muscle, skin, heart, gut, liver, and brain. Adult stem cells typically generate the cell types of the tissue in which they reside and represent a replacement for cells that are lost during an injury or because of disease. Thus, in contrast to embryonic stem cells, adult stem cells have limited differentiation potential.

Although the word “stem cell” was coined over more than a century ago (as shown in the work by Ernst Haeckel in 1868), the first indication of multipotency in stem cells arrived only in the early 1960s, thanks to pioneering studies in hematopoietic stem cells performed by Ernest McCulloch and James Till. Their experiments showed that different blood cells derive from one single type of cell. These cells were able to give rise to “colonies”, where each colony was deriving from one single cell. Moreover, these colony-forming cells could not only “self-renew” but could also specialize in three different blood cell types. Twenty years after, in the early 1980s, Martin Evans, Matthew Kaufmann and Gail Martin showed that murine cells from the ICM could be isolated and cultured outside the body without losing their pluripotency and their ability to self-renew. The cultured murine cells were coined embryonic stem cells (ESCs), because of their ability to mimic the differentiation capacities of ICM cells. The enormous potential of murine ESCs was shown in subsequent work, when it was demonstrated that ESCs could be reintroduced into host blastocysts to give rise to chimeric animals carrying cells derived both from the injected ESCs as well as the host ICM cells. Furthermore, breeding of chimeric animals resulted in offspring carrying only the genetic material of the cultured ESCs, thus indicating that ESCs can contribute to the germline of host animals Finally, in 1998, Thomson and colleagues were able to isolate embryonic stem cells (hESCs) from early human embryos, thereby setting the stage for subsequent efforts to generate distinct cell types for cell replacement therapies in patients. In addition to hESCs, it is now possible to reprogram adult somatic cells to become induced pluripotent stem cells (iPSCs). Human iPSCs were first reported in 2007. iPSCs are adult cells that have been genetically reprogrammed to an embryonic stem cell-like state. Cells from patients can be expanded in culture, differentiated, and reintroduced into the patients. Thus, a source of stem cells can be generated that are specific to the donor, increasing the chance of compatibility. This represents a significant advantage, as immune rejection, which requires a continuous administration of immunosuppressive drugs, and which can occur when non-matched stem cell derivatives are used, may be avoided. Finally, researchers at UCSF generated human pluripotent stem cell lines from blastocysts, thus demonstrating that cells with differentiation capacity into the three germ layers can be isolated from an earlier stage than the ICM stage (Human stem cells from single blastomeres reveal pathways of Embryonic or trophoblast fate specification. (2015); Development, 2015, 142(23):4010-25. doi: 10.1242/dev.122846. Epub 2015 Oct. 19.)

Nowadays, one of the most important potential applications of human pluripotent stem cells, including but not limited to hESCs, iPSCs, blastocyst-derived stem cells, and adult stem cells is the generation of cells and tissues that could be used for cell-based therapies. Stem cells expanded in laboratories are capable of long-term self-renewal: a starting population of stem cells can proliferate for years, yielding to billions of unspecialized cells. Moreover, these stem cells can be differentiated in vitro, and be directed into becoming any specialized cell that can be used to regenerate and repair diseased or damaged tissues in patients. This approach forms the basis of regenerative medicine. Examples of diseases that might be treated by transplanting cells generated from hESCs, iPSCs, or blastocyst-derived and organ stem cells include diabetes, heart disease, Duchenne's muscular dystrophy, traumatic spinal cord injury, and vision and hearing loss. In fact, adult stem cells, such adult hematopoietic stem cells from bone marrow, have already been used in transplants for more than 40 years.

The inventors have made remarkable progress regarding the generation of functional beta cells from human stem cell populations. The underlying strategy is to closely recapitulate the path that pluripotent stem cells take during embryogenesis, from the formation of definitive endoderm, to pancreatic endoderm, to endocrine progenitors, and finally to pancreatic islet cells. These recent advances in the differentiation of human stem cells towards pancreatic islet cells now suggest tangible alternatives to the more conventional treatment options for Type 1 and Type 2 diabetes, the two major forms of diabetes, which affects more than 400 million people worldwide.

In Type 1 diabetes, the insulin-producing beta cells (beta cells) of the pancreas are destroyed by the body's immune system. In Type 2 diabetes, which is the more common form, beta cells are exhausted due to the high insulin demand often arising from insulin resistance in peripheral tissues. This high demand leads to a progressive decline in beta cell function and mass, to the point that the islets can no longer produce enough insulin to overcome the insulin resistance.

Islet transplantation is an effective intervention to restore glucose levels, but the number of cadaveric islets needed is greater than the supply, and patients undergoing islet transplantation require immune suppressive drugs all their life. This is the reason why cell replacement therapy has emerged as a viable alternative to treat diabetes. See, Charles A. Goldthwaite, Jr., Are Stem Cells the Next Frontier for diabetes treatment? In Regenerative Medicine https://stemcells.nih.gov/info/Regenerative_Medicine/2006Chapter7.htm.

Transplantation per se is a challenging procedure, as most of the cells die shortly after transplantation.

To be useful for transplant purposes, stem cells must not only proliferate extensively in culture and differentiate into the desired cell type, but they must also survive in the recipient after transplant and function for the duration of the patient's life. Currently, there is no technology to monitor the health state of transplanted cells after transplantation and over time, with non-invasive readouts.

To achieve an effective strategy for using stem cell therapy, it is critical to develop real-time, practical, efficient, detecting and monitoring systems and methods to monitor the viability, health state, and functional capability of transplanted stem cells.

### 3. Methods

The compositions and systems according to the present disclosure are particularly useful for in vitro, in situ and/or in vivo sensing and real-time monitoring of stem cells during stem cell therapy. The nanoprobes as provided herein are designed to detect miRNAs, or small noncoding RNAs, or mRNAs, which regulate gene expression at post-transcriptional levels, thus serving as early biomarkers. In the presence of target miRNAs, small noncoding RNAs, or mRNAs, the iMS nanoprobes generate a Raman signal, based on a non-enzymatic, strand-displacement process followed by a conformational change of the hairpin oligonucleotide as described herein.

Accordingly, another aspect of the present disclosure provides a method of detecting and monitoring, in real-time, stem cell differentiation in vitro, the method comprising, consisting of, or consisting essentially of; (1) obtaining one or more stem cell-derived cells; (2) transfecting the stem cell-derived cell with an iMS nanoprobe; and (3) detecting the optical signal from nanoprobe.

Physical approaches can be used to directly deliver or transfect drugs or gene probes to desired intracellular locations (e.g., cytosol or nucleus). Among these physical methods, electroporation has been widely used due to its simplicity, uptake effectiveness, less restrictions on probe or cell type, and operation convenience. In electroporation, short, high-voltage electric pulses are applied to surpass the cell membrane capacitance, making the subjected cells transiently permeable. In some embodiments, the stem cell-derived cells are transfected using electroporation.

Another aspect of the present disclosure provides a method of monitoring, in real-time, stem cell transplants in vivo in a subject, the method comprising, consisting of, or consisting essentially of: (1) obtaining one or more stem cell-derived cells; (2) transfecting the stem cell-derived cell with an iMS nanoprobe; (3) administering to the subject the transfected stem cell-derived cell; and (4) detecting the optical signal from the nanoprobe.

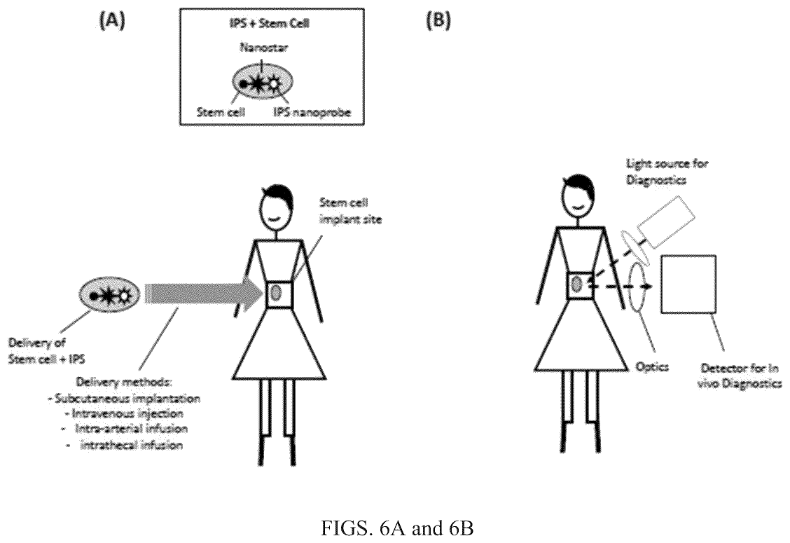

FIG. 6 illustrates one example of a method of in vivo diagnostic modality using compositions and systems according to the present disclosure that can serve as a real time, permanent and continuous “health monitor”. The transfected stem cell-derived cells are administered or introduced (e.g., transplanted) to a subject or patient (FIG. 6A). Depending on specific treatments, stem cell-derived cell delivery methods could include, but are not limited to, subcutaneous implantation with or without synthetic scaffolds, intravenous injection, intra-arterial infusion, and intrathecal infusion. The health status of the transfected stem-cell derived cells can be monitored by the subject and/or by a health care provider using a portable Raman diagnostic system having excitation light source and an optical detector (FIG. 6B). Health status of the transfected stem-cell derived cells can include information regarding viability, functioning capability, and/or health state of the transfected stem-cell derived cells. The one or more stem-cell derived cells can be transfected with one or more nanoprobes. The one or more nanoprobes may comprise inverse molecular sentinels (iMS). The iMS may comprise a plasmonic-active nanoparticle, a stem-loop nucleic acid probe attached at one end to the nanoparticle, the nucleic acid comprising a first sequence (stem-loop probe) and labeled with an optical reporter, and an unlabeled capture placeholder nucleic acid strand comprising a second sequence (placeholder).

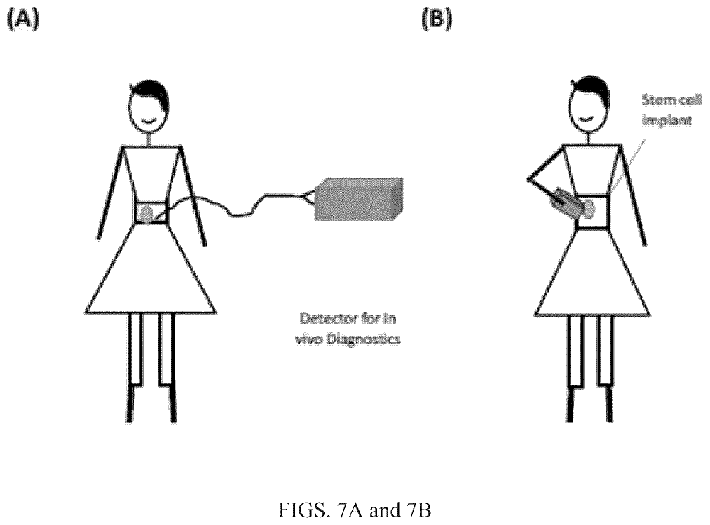

In another embodiment, and as shown in FIG. 7, a portable, pocket-sized Raman diagnostics system with fiberoptics excitation and detection can be used to monitor the transfected stem cell-derived cells (FIG. 7A). Alternatively, a handheld battery-operated Raman reader system can be operated remotely by an iPhone or similar device (FIG. 7B).

It is to be noted that, in addition to the specific embodiments described herein, the compositions, systems, and methods disclosed herein can also be used to monitor many other types of targets in stem cells. Alternate embodiments include other bioreceptors including (but not limited to) aptamers, antibodies, enzymes, cell-based receptors, etc. Other alternate embodiments use chemical receptors, ligands for target recognition and sensing. Other alternate embodiments use chemical sensing species that exhibit a change in Raman/SERS signal when target species or biochemical conditions occur. Some non-limiting examples include pH, O₂, metabolites, chemical ligands, etc. The different aspects of the present disclosure can also be used to monitor the viability, operation, injury, shelf-life for use in a wide variety of medical applications, such as stem and precursor cells from a wide variety of sources (e.g., embryos, gestational and adult tissue), stem cells from reprogrammed differentiated cells, and insulin-producing pancreatic islets.

The SERS detection methods described herein can be combined with other spectroscopic modalities, such as conventional Raman, fluorescence, phosphorescence, absorption sensing and imaging techniques to obtain a more complete status of the health state of the cells being monitored. Some non-limiting examples include the combination of SERS detection of miRNA targets using the sensing platform disclosed herein and conventional Raman detection or imaging. Whereas the sensing platform disclosed herein can monitor the viability, health state, functional capability of a transplanted stem cell system, the Raman technique can provide information on the chemical structure of the cells or chemical species in the cells (proteins, lipids and DNA) according to their vibrational spectra.

The SERS detection methods described herein can be used in other stem cell therapy applications, including the production of tissue and organs. Some examples include tissue-engineered heart muscle, functional tissues and organs, tissue-engineered skin, derived from a patient's own cells, tissue-engineered bladder, derived from a patient's own cells, and small intestinal submucosa (SIS), used to help the body close hard-to-heal wounds. Further examples include tissue-engineered products to induce bone and connective tissue growth, tissue-engineered vascular grafts for heart bypass surgery and cardiovascular disease treatment, and “made-to-order” organs from 3D molecular/organic 3D printing.

Moreover, the disclosed compositions, systems and methods have potential for a wide variety of applications based on DNA/RNA/protein detection, such as biomedical applications, point-of-care diagnostics, quality control applications, global health, cancer research, heart disease diagnostics, and homeland defense.

Furthermore, the disclosed compositions, systems and methods can lead to improved stem cell-based therapeutic uses. A major challenge in stem cell therapy is the survival of these cells in vivo. Currently most of the stem cells die or do not function properly after some periods following transplantation. Currently, the ability to monitor the health state of transplanted cells with non-invasive readouts remains particularly challenging. The proposed sensing platform disclosed herein provides a critical real-time, practical, and efficient nanosystem capable of monitoring the viability, health state, functional capability of transplanted stem cell systems. This sensing capability will greatly improve the efficacy of stem cell-based therapy as the sensing nanoplatform can provide a warning signal of cellular non-functionality that will trigger timely remediation procedures and/or replacement of the non-functioning stem cells when needed.

Other possible novel and additional features using the disclosed methods will be evident to those of skill in the art. Some examples include: the use of the compositions, systems and methods for in vitro and in vivo monitoring of functional properties of stem cell derivatives for cell therapy; stem cell in vivo plasmonics sensing (SC-IPS) probes consisting of metal; SC-IPS probes for multiplex detection; SC-IPS system and methods using multispectral Raman imaging for multiplex detection; SC-IPS system and methods using pulsed laser excitation and time-resolved Raman detection; and SC-IPS system and methods using periodic excitation and phase-resolved Raman detection.

Additionally, the disclosed system and methods can be deployed in several different formats. Some examples include portable in vivo diagnostic system using SC-IPS probes, pocket-sized or palm-sized in vivo diagnostic system using SC-IPS probes, and wrist-watch-sized in vivo diagnostic system using SC-IPS probes.

Another embodiment of the present disclosure provides a method of performing in vivo monitoring using the disclosed systems and methods.

The following Examples are provided by way of illustration and not by way of limitation.

### EXAMPLES

### Example 1: Absorbance Spectra of Gold Nanostar Solutions

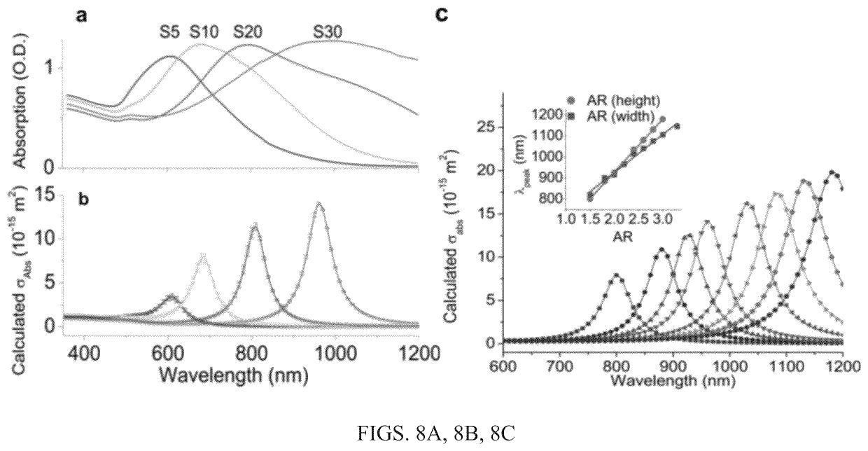

The absorbance spectra of exemplary gold nanostar solutions (S5, S10, S20, and S30) were measured to evaluate use of gold nanostars for deep tissue NIR excitation and absorption in the diagnostic window for stem cell applications. The nanostars increased in size from S5 to S30, with S5 being the smallest and S30 being the largest. FIG. 8(a) is a chart showing an absorbance spectra of exemplary nanostar solutions in citrate buffer (˜0.1 nM in citrate buffer). FIG. 8(b) is a chart showing a corresponding finite element method (FBM)-generated absorption spectra of exemplary nanostars embedded in water. In FIG. 8(b), the solved data points (±1 SD) were interpolated with a spline fit.

FIG. 8(c) is a chart of the scatter plots of polarization-averaged absorption against aspect ratio (AR) tuned by varying branch height while keeping the base width, core and tip diameters and branch number constant. In FIG. 8(c) inset, the linear relationship between plasmon peak position and AR was tuned by varying branch height (circle, R²=0.997) and base width (square, R²=0.987), keeping all other parameters constant.

The nanostars' plasmon peaks are tunable from 600 nm to 1000 nm by adjusting the Ag+ concentration. This is accompanied by a visible change in the solution color during synthesis from dark blue to dark gray as the plasmon red-shifts and broadens. Both the plasmon peak position and spectral width followed a linear trend with increasing Ag+ concentration. A plateau was reached around an Ag+ concentration of 30 μM. These results show that nanostars can be synthesized in a controlled fashion and exploited as potential candidates for deep tissue NIR excitation and absorption in the diagnostic window for stem cell applications.

### Example 2: Synthesis of Exemplary Plasmonic Gold Nanostars

AuNS were synthesized using known methods. A 12-nm gold seed solution was prepared by adding 15 mL of 1% trisodium citrate to 100 mL of a boiling solution of 1 mM HAuCl4. The solution was kept boiling for an additional 15 minutes, cooled to room temperature in an ice bath, filtered through a 0.22-μm nitrocellulose membrane, and stored at 4° C. until use. To produce the larger AuNS (designated S30), 100 μL of the gold seed was added to a 10 mL solution of 0.25 mM HAuCl₄ containing 10 μL of 1 N HCl, immediately followed by the simultaneous addition of 50 μL 0.1 M AA and 100 μL 3 mM AgNO₃ under moderate stirring. The smaller AuNS (designated S5) were produced in the same manner as above but using 0.5 mM AgNO₃ in place of 3 mM AgNO₃.

Nanostars were then produced by reducing tetrachloroauric acid onto 12-nm citrate-stabilized gold seeds in an acidic environment using a weak reducing agent, ascorbic acid (AA), and stabilizing with sodium citrate. The growth of nanostars on seeds was completed in less than half a minute. It was a simple and rapid method. The particles were stable at 4° C. for at least a week after centrifugal washing.

The exemplary nanostars produced the most red-shift plasmon under lower pH, higher vortexing speed and AA/HAuCl₄ ratio of approximately 1.5-2. The concentrations of HAuCl₄ and seeds were selected such that nanostar sizes were around 60 nm. It was observed that silver ions play a role in controlling the formation of nanostar geometry. Without Ag+, the resulting particles were polydisperse in both size and shape. The addition of a small amount of Ag+ led to a high yield of monodisperse star-shaped particles. The particle diameters of the synthesized nanoparticles were within approximately 50-70 nm. It is believed that Ag+ assist the anisotropic growth of Au branches on certain crystallographic facets on multitwinned citrate seeds.

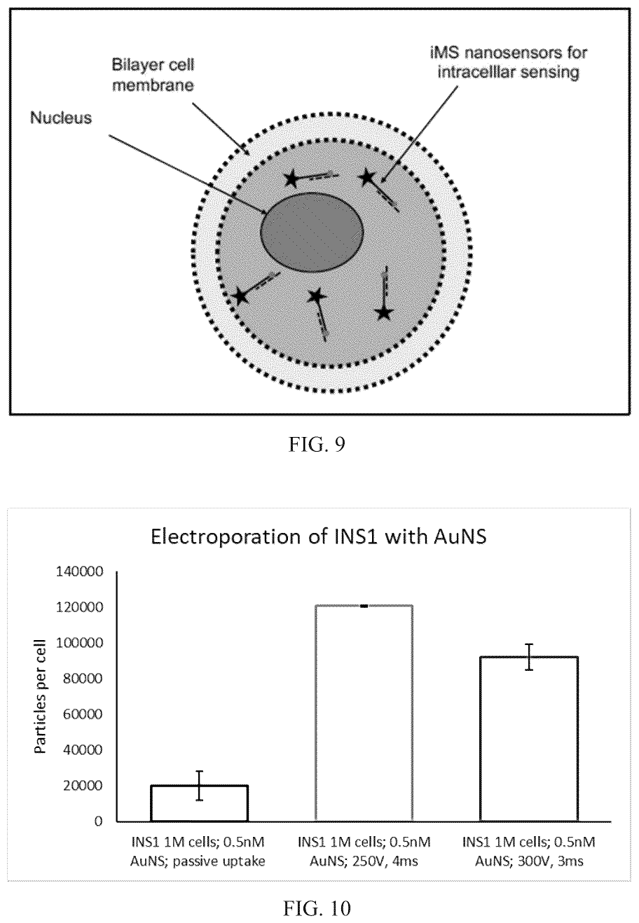

A schematic diagram of an exemplary embodiment of an intracellular molecular nanoprobe is shown in FIG. 9, which depicts iMS nanosensors for intracellular sensing inside a stem cell.

### Example 3: Transfection of AuNS-PEG into INS1 Beta Cells by Electroporation

In an exemplary method, AuNS-PEG was transfected into cells of INS1 cell line (i.e., a rat insulinoma cell line) using electroporation. Uptake of AuNS-PEG through electroporation resulted in a 6-fold enrichment compared to passive uptake, when using 0.5 nM particles, one million cells, and electroporation at 250V with 4 ms pulse length.

To perform electroporation, one million INS1 cells were resuspended in 800 uL OptiMEM media containing 0.5 nM AuNS-PEG. Electroporation was performed using a 4-cm cuvette and carried out at room temperature using either 250V with 4 millisecond (ms) pulse length or 300V with 3 ms pulse length. After electroporation, the cuvette was immediately placed in the incubator (37° C., 5% CO₂) for 15 minutes. The cells were washed in PBS by centrifugation three times to remove free nanoparticles in solution (300 g, 3 min) before being resuspended in cell culture media and then added to a single well in a 6-well plate. Cells were placed in the incubator for 12 hours, after which cells were washed with PBS and trypsinized to be collected for ICP-MS.

For passive uptake, one million INS1 cells were plated in a single well of a 6-well plate and 0.5 nM AuNS-PEG was allowed to incubate with the cells for 12 hours. Following incubation, cells were washed 3 times with PBS to remove any particles in the media and trypsinized to be collected for ICP-MS. FIG. 10 is a bar chart comparing transfection results for passive uptake and uptake using electroporation at 250V with 4-ms pulse length and 300V with 3-ms pulse length. As can be seen, transfection using electroporation at 250V with 4-ms pulse length resulted in the highest uptake.

Studies were performed to investigate the effect of electroporation on the configuration of inverse molecular sentinels (iMS).

One million INS1 cells were suspended in 800 μL OptiMEM media containing 0.15 nM AuNS-iMS in the off position (OFF). The 800 μL solution was added to a 4-cm electroporation cuvette. Electroporation was carried out at room temperature using either 250 V with 4-ms pulse length or 300 V with 3-ms pulse length. Following electroporation, the cuvette was immediately sprayed with ethanol and placed in the incubator (37° C., 5% CO₂) for 15 minutes. The cells were washed in PBS by centrifugation three times to remove free nanoparticles in solution (300 g, 3 min) before being resuspended in cell culture media and added to a glass dish. Cells were placed in the incubator for 12 hours, after which Raman imaging measurements were performed using 633-nm laser excitation.

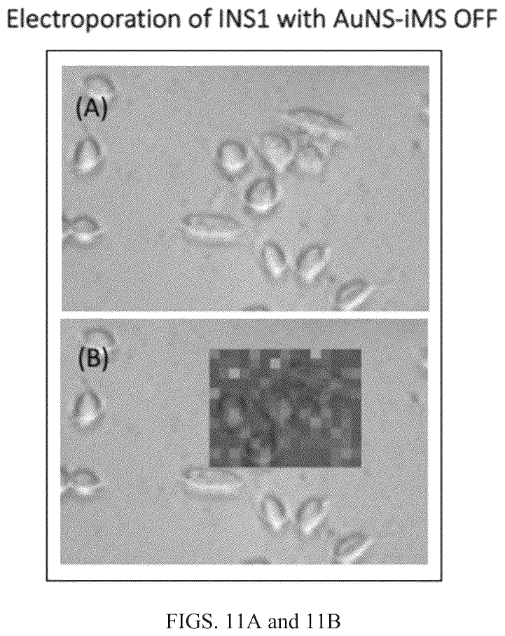

It was found that electroporation of INS1 with non-targeting AuNS-iMS in the OFF position (+ placeholder) did not produce an ON signal within cells, indicating that the electroporation process did not alter the iMS probes. FIG. 11A is an image of the INS1 cells after transfection with AuNS-iMS using electroporation. FIG. 11B is an image showing a Raman spectral imaging map (false color) measured at the 557 cm⁻¹ Raman band. The results indicate that the iMS signal remained “OFF” following electroporation.

A similar analysis was performed using AuNS-iMS ON. INS1 transfected with AuNS-iMS ON (i.e., no placeholder; iMS was turned on by adding target prior to electroporation) using electroporation was evaluated to determine if there was an effect on the iMS ON.

1E6 INS1 cells were suspended in 800 μL OptiMEM media containing 0.15 nM AuNS-iMS ON. The 800 μL was added to a 4-cm electroporation cuvette. Electroporation was carried out at room temperature using either 250V with 4 ms pulse length or 300 V with 3 ms pulse length. After electroporation, the cuvette was immediately sprayed with ethanol and placed in the incubator (37° C., 5% CO₂) for 15 minutes. The cells were washed in PBS by centrifugation three times to remove free nanoparticles in solution (300 g, 3 min) before being resuspended in cell culture media and added to a glass dish. Cells were placed in the incubator for 12 hours, after which Raman imaging measurements were performed using 633-nm laser excitation.

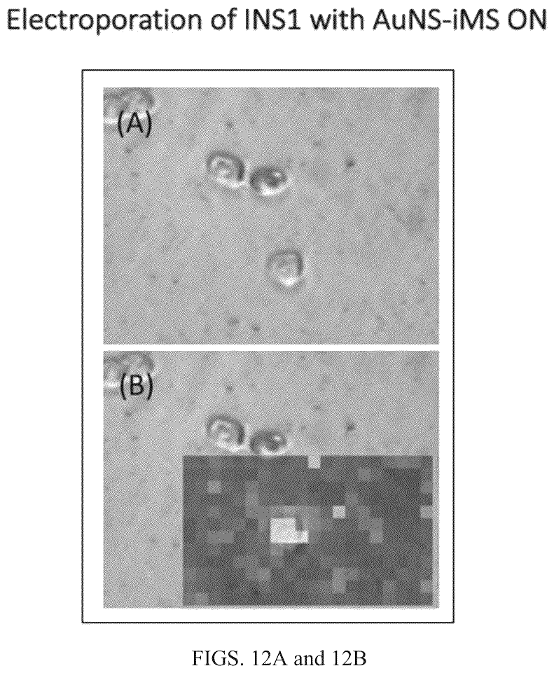

FIG. 12A is an image of the cells after electroporation. FIG. 12B shows the Raman spectral imaging map (false color) measured at the 557 cm⁻¹ Raman band. The results in FIG. 12B indicate that iMS signal remained “ON” following electroporation.

Raman measurements were performed with iMS nanoprobes for miR200b following electroporation into INS1 cells. One million INS1 cells were suspended in 788 μL OptiMEM media. 12 μL of 10 nM iMS OFF (+placeholder) or ON (turned on prior) were added to cell solution. The 800 μL solution was added to a 4-cm electroporation cuvette. Electroporation was carried out at room temperature using 250 V with pulse length of 10 ms. Following electroporation, the cuvette was immediately sprayed with ethanol and placed in the incubator (37° C., 5% CO₂) for 15 minutes. The cells were washed in PBS by centrifugation three times to remove free nanoparticles in solution (300 g, 3 min) before being resuspended in 100 μL PBS for Raman measurements (633-nm laser excitation, 1% power, 10-sec data accumulation time).

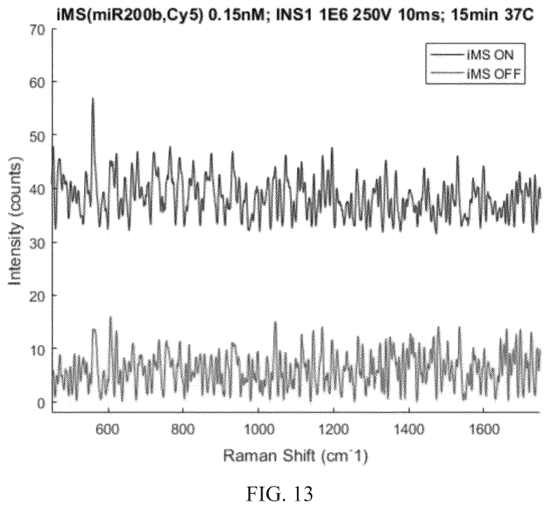

For each experiment, three SERS measurements were performed per sample and averaged into a single spectrum. The SERS spectra were background subtracted and smoothed using a Savitsky-Golay filter (five-point window and first-order polynomial). Following measurements, cells were plated in a six well plate and placed back in the incubator. FIG. 13 shows the presence of the Raman peak at 558 cm⁻¹ associated to the iMS probe with iMS “ON” (Upper curve) and no peaks for iMS OFF (Lower curve).

### Example 4: Monitoring CEL-miR39 (Cy5) Nanoprobes

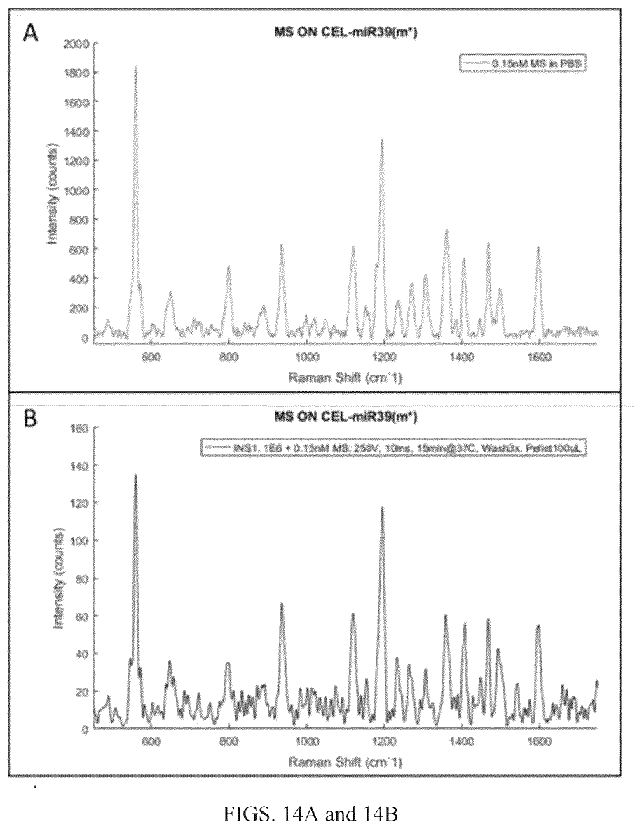

To progress towards monitoring iMS within cells throughout the stem cell differentiation process, a pseudo-sensor was developed. The sensor is designed for CEL-miR-39 (a sequence that is commonly used as a spike-in control for RT-qPCR) with modified bases (2′-O-methoxy-ethyl bases and phosphorothioate (PS) linkages) that are resistant to single stranded binding proteins (SSB) and nuclease degradation. This probe has a stem with a higher melting temperature than the unmodified probe. For these reasons, it was anticipated that the probe would remain in the ‘ON’ position throughout differentiation. FIG. 14A shows the SERS spectrum of pure CEL-miR39 (Cy5) nanoprobes in solution. FIG. 14B shows the SERS spectrum of CEL-miR39 (Cy5) nanoprobes after being transfected into INS1 cells.

The curve of FIG. 14A shows the SERS spectrum of the CEL-miR-39 MS probe (with Cy5 label) at concentration of 0.15 nM in PBS (633-nm laser excitation, 1% power, 10-sec accumulation). The curve in FIG. 14B represents the SERS spectrum acquired after the CEL-miR-39 MS probe (with Cy5 label) was electroporated into INS1 cells.

One million INS1 cells were suspended in 788 μL OptiMEM media. 12 μL of 10-nM CEL-miR-39 MS ON was added to cell solution. The 800-μL solution was added to a 4-cm electroporation cuvette. Electroporation was carried out at room temperature using 250V with pulse length of 10 ms. After electroporation, the cuvette was immediately sprayed with ethanol and placed in the incubator (37° C., 5% CO₂) for 15 minutes. The cells were washed in PBS using centrifugation three times to remove free nanoparticles in solution (300 g, 3 minutes) before being resuspended in 100 μL PBS for Raman measurements (633-nm excitation, 1% power, 10 sec accumulation).

For each experiment, three SERS measurements were performed per sample and averaged into a single spectrum. All SERS spectra reported herein were background subtracted and smoothed using a Savitsky-Golay filter (five-point window and first-order polynomial). Following measurements, cells were plated in a six well plate and placed back in the incubator.

Table 1 shows results of a viability study of electroporated INS1 cells. One million INS1 cells were suspended in 800-μL OptiMEM media.

TABLE 1
Viability Study of Electroporated INS1 Cells

| | | | | |
|---|---|---|---|---|
| Voltage | Pulse Length | Pulse# | #Live Cells | % of original 1E6 |

WITH AuNS-PEG (1.5 nM); 15 min @ 37° C. post electroporation

| | | | | | |
|---|---|---|---|---|---|
| 250 V | 10 | ms | 1 | 4.87E+05 | 49% |
| 300 V | 4 | ms | 1 | 1.53E+05 | 15% |

| | | |
|---|---|---|
| No | 4.12E+05 | 41% |
| electroporation | | |

WITHOUT PARTICLES; 2 min recovery @ 37° C. b/t pulses; 15 min @ 37° C. post
electroporation

| | | | | | |
|---|---|---|---|---|---|
| 250 V | 0.5 | ms | 3 | 4.28E+05 | 43% |
| | | | 6 | 5.53E+05 | 55% |
| | 3 | ms | 1 | 3.61E+05 | 36% |
| | | | 2 | 2.82E+05 | 28% |
| | 10 | ms | 1 | 3.84E+05 | 38% |
| | | | 2 | 2.90E+05 | 29% |

The cell solution was added to a 4-cm electroporation cuvette. Electroporation was carried out at room temperature using 250V with pulse lengths of 0.5, 3, or 10 ms. If replicate pulses were performed, cells were allowed to recover at 37° C. for 2 min. After electroporation was complete, the cuvette was immediately sprayed with ethanol and placed in the incubator (37° C., 5% CO₂) for 15 minutes. The cells were washed in PBS by centrifugation three times (300 g, 3 min) before being resuspended in culture medium and plated in a six well plate. Cells were placed in the incubator for 12 hours before assessing viability. Cells were trypsinized and centrifuged to collect a pellet. The pellet was dispersed in 1 mL cell culture media. Trypan blue dye was used to assess viability during counting.

### Example 5. Retention of AuNS-PEG in hESCs

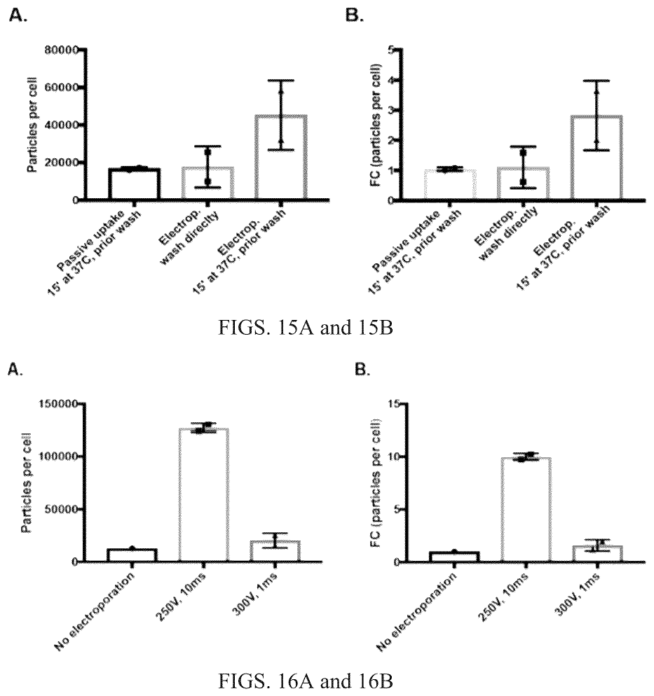

Uptake and retention of AuNS-PEG in hESCs were measured. Table 2 and FIGS. 15-18 illustrate the results of this study. The viability of hESCs 24 hours after electroporation at varying voltages, pulse lengths and number of pulses performed was measured. The results are shown in Table 2. As the results in Table 2 show, viability of hESCs after electroporation was adversely affected by voltages higher than 300 V, pulse lengths longer than 10 ms, and by the number of pulses performed subsequently.

TABLE 2
Viability of hESCs 24 h post electroporation

| Electoporation | |
|---|---|
| conditions | % live cells |
| 300 V, 1 ms, 1pulse | 100% |
| 250 V, 3 ms, 1pulse | 100% |
| 300 V, 3 ms, 1pulse | 100% |
| 350 V, 1 ms, 1pulse | 95% |
| 350 V, 3 ms, 1pulse | 70% |
| 250 V, 3 ms, 2pulse | 70% |
| 250 V, 10 ms, 1pulse | 70% |
| 500 V, 1 ms, 1pulse | 10% |
| 300 V, 3 ms, 2pulse | 10% |
| 300 V, 10 ms, 1pulse | 10% |
| 500 V, 3 ms, 1pulse | 5% |
| 350 V, 10 ms, 1pulse | 5% |
| 250 V, 10 ms, 2pulse | 5% |
| 500 V, 0.5 ms, 6pulse | 0% |
| 350 V, 3 ms, 2pulse | 0% |
| 300 V, 10 ms, 2pulse | 0% |
| 350 V, 10 ms, 2pulse | 0% |

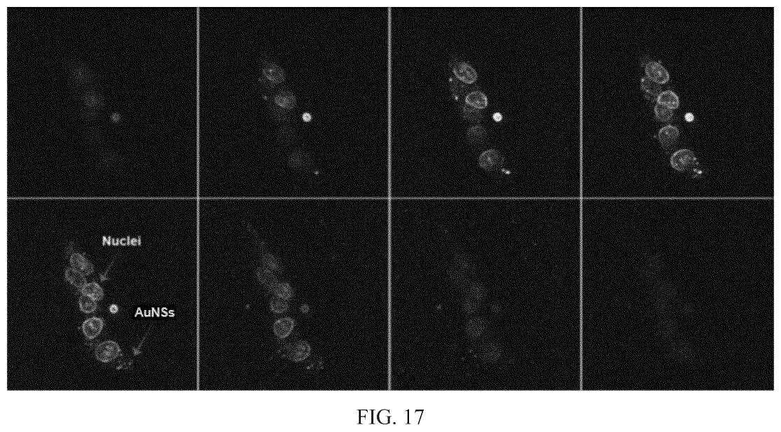

Electroporation efficiency (measured as number of gold nanoparticles taken up per cell) was found to be improved by increasing the length of the pulse, rather than the voltage (see, e.g., FIGS. 16A and B). The optimal condition was determined to be 250 V for 10 ms, using one single pulse (see, e.g., FIG. 17). FIGS. 16A and 16B are charts showing ICP/MS quantification of AuNSs in hESCs immediately after electroporation (0.15 nM AuNSs, PBS). FIG. 16A and FIG. 16B illustrate the same data in different formats. FIG. 16A shows the absolute number of particles per cell, whereas FIG. 16B shows the calculated fold change.

FIG. 17 provides multiphoton z-stack images of hESC cells 24 h after electroporation with 0.15 nM AuNSs.

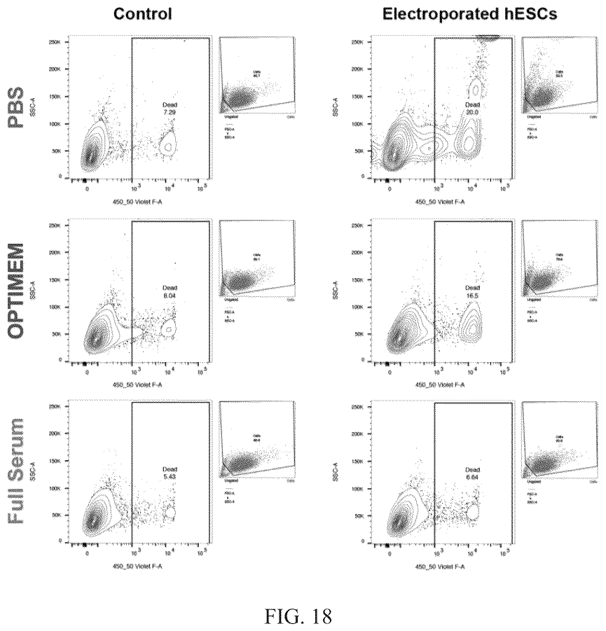

A balance between cell viability and electroporation efficiency was found by optimizing electroporation media. Although electroporation in PBS increased the number of particles per cell up to 10 folds (compared to passive uptake, see FIG. 16), viability of hESCs right after electroporation was decreased, when compared to electroporation performed in OPTIMEM with reduced serum. This result is shown in FIG. 18, which includes images of flow cytometry analysis of hESCs stained with DAPI 30 minutes after electroporation.

Electroporation in OPTIMEM with reduced serum showed up to 4-fold increase of AuNSs, compared to passive uptake, and up to 60K AuNSs per cell, 48 hours after electroporation (see FIG. 16). High electroporation efficiency was reached when hESCs were rested for 15 min at 37° C. prior to washing the cells in PBS to remove the excess particles (not uptaken). Washes performed directly after electroporation caused a complete loss of uptake enrichment (see FIG. 15), likely due to inefficient pore closure. FIG. 15A and FIG. 15B are charts showing ICP/MS quantification of AuNSs in hESCs 48 hours after electroporation (0.15 nM AuNSs, OPTIMEM). FIG. 15A and FIG. 15B illustrate the same data in different formats. FIG. 15A shows the absolute number of particles per cell, whereas FIG. 15B shows the calculated fold change.

Full serum media gave the highest cell viability, but higher cell viability reflects a decrease in pore formation and AuNSs uptake.

Retention of AuNSs was tested during 20 days of differentiation of hESCs into pancreatic beta cells. The differentiation protocol began on the first day of differentiation and was based on the formation of 3D cell clusters (spheres) starting from a 2D culture.

The differentiation protocol of hESCs into pancreatic beta cells used the following protocol. Human embryonic stem cells (hESCs) were maintained and propagated on mouse embryonic fibroblasts (MEFs) in hESC media (DMEM F-12 supplemented with 1× Glutamax, 1×MEME-NEAA, 1× Betamercaptoethanol, KSR and FGF-2). Confluent hESC cultures were dissociated into single-cell suspension and seeded in suspension plates in hPSC media supplemented with Activin A (10 ng/ml, R&D Systems) and HeregulinB (10 ng/ml, Peprotech). Plates were incubated on an orbital shaker to induce 3D sphere formation. Spheres were cultured for 20 days using the following media:
Day 1: RPMI (Gibco) containing 0.2% FBS, 1:5,000 ITS (Gibco), 100 ng/ml activin A, 50 ng/ml WNT3a (R&D Systems)
Day 2: RPMI containing 0.2% FBS, 1:2,000 ITS, 100 ng/ml activin A
Day 3: RPMI containing 0.2% FBS, 1:1,000 ITS, 2.5 μM TGFbi IV (CalBioChem), 25 ng/ml KGF (R&D Systems)
Day 4-5: RPMI containing 0.4% FBS, 1:1,000 ITS, 25 ng/ml KGF
Day 6-7: DMEM (Gibco) with 25 mM glucose containing 1:100 B27 (Gibco), 3 nM TTNBP (Sigma)
Day 8: DMEM with 25 mM glucose containing 1:100 B27, 3 nM TTNBP, 50 ng/ml EGF (R&D Systems)
Day 9-11: DMEM with 25 mM glucose containing 1:100 B27, 50 ng/ml EGF, 50 ng/ml KGF
Day 12-20: DMEM with 25 mM glucose containing 1:100 B27, 1:100 Glutamax (Gibco), 1:100 NEAA (Gibco), 10 μm ALKi II (Axxora), 500 nM LDN-193189 (Stemgent), 1 μm Xxi (Millipore), 1 μM T3 (Sigma-Aldrich), 0.5 mM Vitamin C, 1 mM N-acetyl Cysteine (Sigma-Aldrich), 10 μM zinc sulfate (Sigma-Aldrich) and 10 μg/ml of Heparin sulfate.

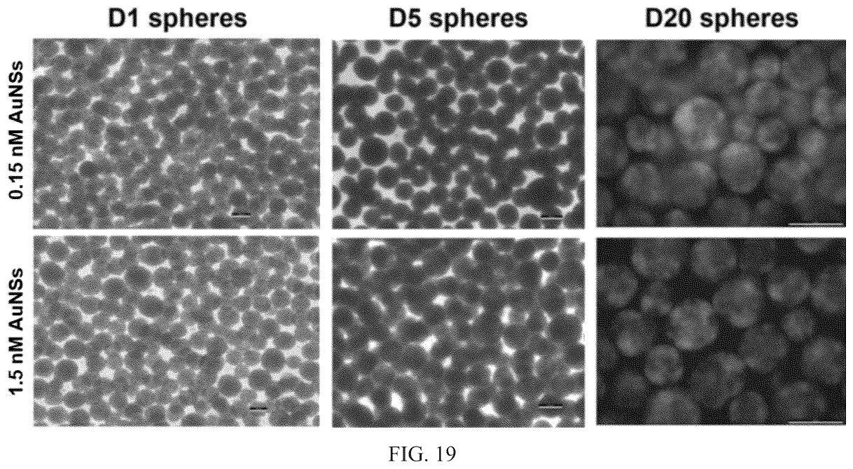

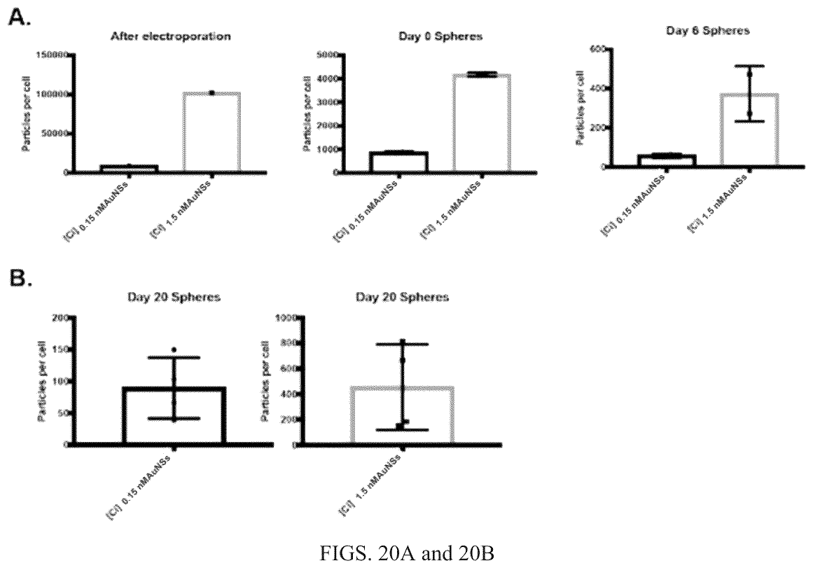

FIG. 19 provides bright field images and fluorescence images of clusters at Day 1, Day 5, and Day 20 of differentiation and shows that hESCs electroporated with AuNSs can form spheres. All clusters contained AuNSs. FIGS. 20A and 20B provide charts showing that hESCs electroporated with AuNSs retained AuNSs until the end of the differentiation protocol.

Due to the high level of cell proliferation occurring at different stages of differentiation, the number of particles per cell was quickly diluted into daughter cells. To counteract this loss, two different concentrations of AuNSs were tested: 0.15 nM and 1.5 nM. The highest concentration of AuNSs did not show toxicity and led to the formation of D20 spheres containing up to 800 particles per cell (see, e.g., FIG. 20). Each sphere contained between 5000 and 8000 pancreatic progenitor cells. Thus, each sphere may have contained between 4 and 6.6 million AuNSs.

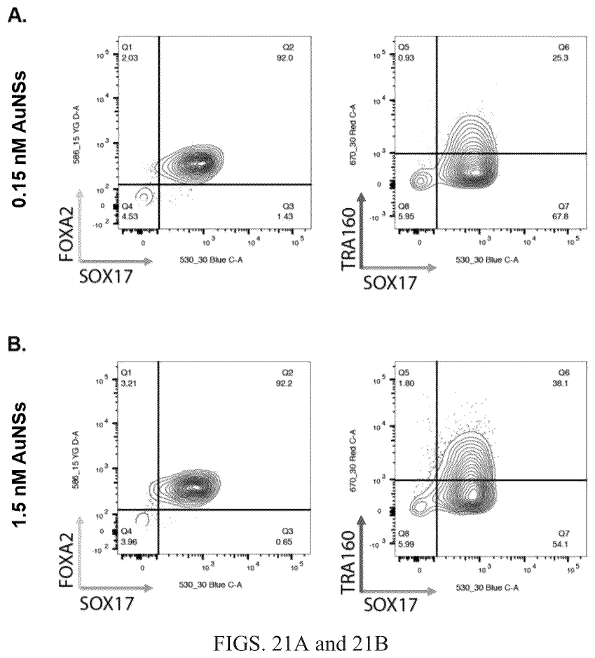

Interference of the AuNSs with the differentiation potential of hESCs into beta cells was monitored by intracellular staining of markers expressed during the formation of endoderm and at the final pancreatic beta cell stage. Specifically, on day 2 intracellular staining was performed to quantify the percentage of cells double positive for endoderm markers FOXA2 and SOX17, and negative for the stem cell marker TRA160. On day 20, intracellular staining was performed to quantify the percentage of cells expressing PDX1, a marker for pancreatic progenitor cells, and to quantify the percentage of cells double positive for NKX6.1 and INS, which are markers of beta cells. Data showed no inhibitory effect of AuNSs on the formation of endoderm, as observed by the presence of >90% of cells double positive for FOXA2 and SOX17, and loss of TRA160. FIG. 21 provides images of flow cytometry analysis of Day 2 spheres electroporated with 0.15 nM AuNSs (A) or 1.5 nM AuNSs (B).

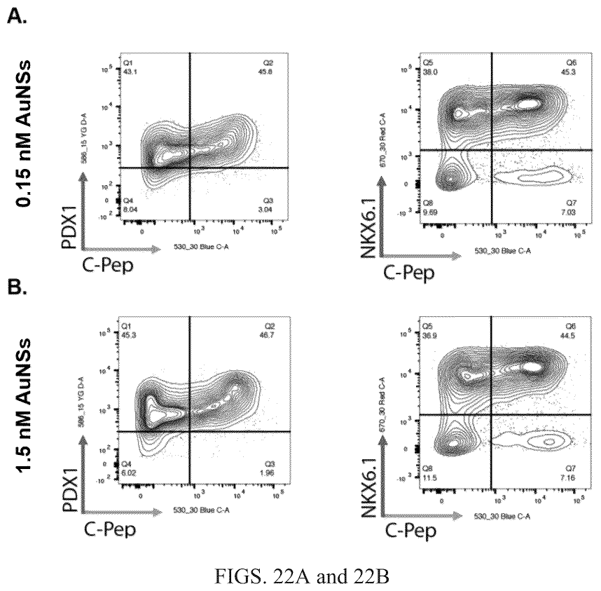

FIG. 22 provides images of flow cytometry analysis of Day 20 spheres electroporated with 0.15 nM AuNSs (A) or 1.5 nM AuNSs (B). As can be seen in FIG. 22, there was no effect on the formation of pancreatic beta cells, as observed by the presence of >90% of the population expressing PDX1 and more than 40% of the population co-expressing C-peptide and NKX6-1. The presence of higher levels of AuNSs (1.5 nM versus 0.15 nM) also had no effect on the differentiation potential.

One skilled in the art will readily appreciate that the present disclosure is adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. It will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

That which is claimed is:

1. An in vivo method of monitoring viability of stem cell-derived cells used in stem cell therapy, comprising
introducing one or more stem cell-derived cells having one or more nanoprobes to a subject, wherein said one or more nanoprobes are configured to provide health status information for the one or more stem cell-derived cells; and
detecting an optical signal from the one or more nanoprobes after introduction of the one or more stem cell-derived cells to the subject.

2. The method of claim 1, wherein health status information for the one or more stem cell-derived cells having one or more nanoprobes comprises information regarding viability, functioning capability, and/or health state of the one or more stem-cell derived cells.

3. The method of claim 1, wherein the one or more nanoprobes comprise inverse molecular sentinels (iMS), comprising:
at least one plasmonic-active nanoparticle,
a stem-loop nucleic acid probe attached at one end to the nanoparticle, the nucleic acid comprising a first sequence (stem-loop probe) and labeled with an optical reporter, and
an unlabeled capture placeholder nucleic acid strand comprising a second sequence (placeholder).

4. The method of claim 3, wherein the unlabeled capture placeholder nucleic acid strand comprises a nucleotide sequence designed to hybridize to and capture a nucleic acid target of interest.

5. The method of claim 4, wherein the nucleic acid target of interest comprises microRNAs, small noncoding RNAs, mRNAs, or DNA sequences.

6. The method of claim 1, wherein the one or more nanoprobes comprise a bioreceptor, which comprises a nucleotide sequence, an aptamer, an antibody, an enzyme, or a cell-based receptor to capture molecular species of interest.

7. The method of claim 1, wherein the one or more nanoprobes comprise a chemical receptor or a ligand for target recognition and sensing.

8. The method of claim 1, wherein the optical signal is a Raman signal or surface-enhanced Raman scattering (SERS) signal.

9. The method of claim 1, wherein the one or more stem-cell derived cells having one or more nanoprobes are introduced to the subject via subcutaneous implantation with or without synthetic scaffolds, intravenous injection, intra-arterial infusion, or intrathecal infusion.

10. The method of claim 1, wherein the one or more stem cell-derived cells comprise a stem cell.

11. The method of claim 1, wherein detecting is performed using a fiber optics-based readout system.

12. The method of claim 11, wherein the readout system is monitored by the subject and/or by a health care provider.

13. The method of claim 11, wherein the readout system is portable.

14. The method of claim 13, wherein the readout system is handheld.

15. A method of monitoring viability of stem cell-derived cells, comprising
introducing one or more stem cell-derived cells to a cell culture media,
introducing one or more nanoprobes to the cell culture media, whereby the one or more stem cell-derived cells are transfected with the one or more nanoprobes, and
detecting an optical signal from the one or more nanoprobes after transfection.

16. The method of claim 15, wherein the method of monitoring viability of stem-cell derived cells comprises monitoring operation, injury, and/or shelf-life of the stem-cell derived cells for use in one or more of stem and precursor cells, stem cells from reprogrammed differentiated cells, and insulin-producing pancreatic islets.

17. The method of claim 16, wherein the stem and precursor cells are from sources such as embryos, gestational cells, and adult tissue.

18. The method of claim 15, wherein the one or more stem cell-derived cells are introduced to the cell culture media before, after, or simultaneously with introduction of the one or more nanoprobes to the cell culture media.

19. The method of claim 15, wherein the one or more nanoprobes comprise inverse molecular sentinels (iMS), comprising:
at least one plasmonic-active nanoparticle,
a stem-loop nucleic acid probe attached at one end to the nanoparticle, the nucleic acid comprising a first sequence (stem-loop probe) and labeled with an optical reporter, and
an unlabeled capture placeholder nucleic acid strand comprising a second sequence (placeholder).

20. The method of claim 19, wherein the unlabeled capture placeholder nucleic acid strand comprises a nucleotide sequence designed to hybridize to and capture a nucleic acid target of interest.

21. The method of claim 20, wherein the nucleic acid target of interest comprises microRNAs, small noncoding RNAs, mRNAs, or DNA sequences.

22. The method of claim 3, wherein the first sequence (stem-loop probe) comprises the nucleotide sequence AAAAACTAAGAAAAAAAAATGGCAGTGTCTTAG (miR-34a stem loop probe; SEQ ID NO: 1) and the second sequence (placeholder) comprises the nucleotide sequence ACAACCAGCTAAGACACTGCCATTTT (miR-34a placeholder; SEQ ID NO: 2) for miR-34a miRNA sensing.

23. The method of claim 3 or 19, wherein the first sequence (stem-loop probe) comprises the nucleotide sequence AAAAATACCCTTTATATAAAAATAATACTGCCGGGTA (miR-200b-3p stem-loop probe; SEQ ID NO: 3) and the second sequence (placeholder) comprises the nucleotide sequence TCCATCATTACCCGGCAGTATTATTTT (miR200b-3p placeholder; SEQ ID NO: 4) for miR200b-3p miRNA sensing.

24. The method of claim 3 or 19, wherein the first sequence (stem-loop probe) comprises the nucleotide sequence AAAAAACCCAAATAAAAAATAATACTGCCGGGT (miR200c-3b stem-loop probe; SEQ ID NO: 5) and the second sequence (placeholder) comprises the nucleotide sequence TCCATCATTACCCGGCAGTATTA (miR200c-3p; SEQ ID NO: 6) for miR200c-3p miRNA sensing.

25. The method of claim 3 or 19, wherein the first sequence (stem-loop probe) comprises the sequence SH-AAAAA+CT+AA+GA+AA+AA+AA+AA+TG+GC+GC+AG+TG+TC+TT+AG+(miR-34a; SEQ ID NO: 7) and labeled with a Raman reporter; (2) a plasmonic-active nanoparticle; and (3) an unlabeled capture placeholder nucleic acid strand comprising a second sequence, the sequence comprising AC+AA+CC+AG+CT+AA+GA+CA+CT+GC+CA+TT+TT (MIR-34a; SEQ ID NO: 8) for miR-34a miRNA sensing.

26. The method of claim 3, wherein the plasmonic-active nanoparticle is selected from the group consisting of silver nanospheres, gold nanospheres, silver nanoshells, gold nanoshells, silver nanostars and gold nanostars.

27. The method of claim 3, wherein the optical reporter is selected from the group consisting of: Raman dye, 3,3′-Diethylthiadicarbocyanine iodide (DTDC), 3,3′-diethylthiatricarbocyanine iodide (DTTC), 1,1′,3,3,3′,3′-Hexamethylindotricarbocyanine iodide (HITC), CY3 dye, CY3.5 dye, CY5.5 dye, CY7 dye, CY7.5 dye, a positively-charged hydrophobic near infrared (NIR) dye, IR-780, IR-792, IR-797, IR-813, methylene blue hydrate (MB), 4-mercaptobenzoic acid (4-MBA), 5,5′-dithiobis-2-nitrobenzoic acid (DTNB), 4-aminothiophenol (4ATP), fluorescein, fluorescein isothiocyanate (FITC), thionine dyes, rhodamine-based dye, crystal violet, a fluorescence label, or absorbance label.

28. The method of claim 15, wherein transfection comprises electroporating the cell culture media containing the one or more stem cell-derived cells and the one or more nanoprobes; wherein at least a portion of the one or more stem cell-derived cells remain viable after electroporation.

29. The method of claim 28, wherein at least 35% of the one or more stem cell-derived cells remain viable after electroporation.

30. The method of claim 28, wherein 30%-80% of the one or more stem cell-derived cells remain viable after electroporation.

31. The method of claim 28, wherein electroporation is carried out at 200V-500V, such as at 250 V or at 300 V.

32. The method of claim 28, wherein electroporation is carried out with a pulse length of 0.5-10 milliseconds (ms) or 3-5 milliseconds (ms) or 3-4 milliseconds (ms).

33. The method of claim 28, wherein electroporation is carried out at 250V with a pulse length of 2-4 milliseconds (ms).

34. The method of claim 28, wherein electroporation is carried out at 300V with a pulse length of 2-4 milliseconds (ms).

35. The method of claim 28, wherein electroporation is carried out with 1-5 pulses or 1-3 pulses or 1 pulse.

36. The method of claim 15, further comprising administering the one or more transfected stem cell-derived cells to a subject and detecting the optical signal from the one or more nanoprobes in vivo.

37. The method of claim 36, wherein monitoring the viability of the one or more stem cell-derived cells in vivo is performed real-time.

38. The method of claim 15, wherein monitoring the viability of one or more stem cell-derived cells is performed real-time to monitor stem cell differentiation in vitro.

39. The method of claim 1 or 15, wherein the one or more stem cell-derived cells comprise a stem cell.

40. The method of claim 1 or 15, wherein the one or more stem cell-derived cells are selected from the group consisting of beta cells, cardiomyocytes, neural cells, hepatocytes, renal cells, epithelial cells, endothelial cells, and combinations thereof.

41. A method of transfecting stem cell-derived cells with nanoprobes using electroporation while maintaining a configuration of at least a portion of the nanoprobes, comprising
providing a cell culture media comprising one or more stem cell-derived cells and one or more nanoprobes having initial configurations, and
electroporating the cell culture media containing the one or more stem cell-derived cells and one or more nanoprobes; wherein at least a portion of the one or more nanoprobes maintain their initial configurations after electroporation.

42. The method of claim 41, wherein the one or more nanoprobes comprise inverse molecular sentinel (iMS) nanoprobes.

43. The method of claim 42, wherein the initial configurations of the iMS nanoprobes are OFF.

44. The method of claim 42, wherein the initial configurations of the iMS nanoprobes are ON.

45. A method of increasing uptake of nanoprobes into stem cell-derived cells while maintaining viability of the stem cell-derived cells, comprising:
providing a cell culture media comprising one or more stem cell-derived cells and one or more nanoprobes, and
electroporating the cell culture media containing the one or more stem cell-derived cells and the one or more nanoprobes; whereby an amount of the one or more nanoprobes transfected into the one or more stem-cell derived cells is greater than an amount that would have transfected into the one or more stem-cell derived cells if transfection consisted of only passive uptake.

46. The method of claim 45, wherein at least 35% of the one or more stem cell-derived cells remain viable after electroporation.

47. The method of claim 45, wherein 30%-80% of the one or more stem cell-derived cells remain viable after electroporation.

48. The method of claim 41 or 45, wherein the one or more stem cell-derived cells comprise a stem cell.

49. The method of claim 45, wherein the one or more nanoprobes comprise inverse molecular sentinel (iMS) nanoprobes.

50. The method of claim 42, wherein the iMS nanoprobes comprise:
a plasmonic-active nanoparticle,
a stem-loop nucleic acid probe attached at one end to the nanoparticle, the nucleic acid comprising a first sequence (stem-loop probe) and labeled with an optical reporter, and
an unlabeled capture placeholder nucleic acid strand comprising a second sequence (placeholder).
